(19) United States
(12) Patent Application Publication
(10) Pub. No.: US 2020/0229744 A1
(43) Pub. Date: Jul. 23, 2020

(54) DEVICE FOR THE IMPROVED COLLECTION OF CORD BLOOD FROM AN UMBILICAL CORD

(71) Applicant: AMERICORD REGISTRY LLC, New York, NY (US)

(72) Inventors: Andrew L'Huillier, Edison, NJ (US); Ayanna Bryan, Bloomfield, NJ (US); Martin Smithmyer, New York, NJ (US)

(73) Assignee: AMERICORD REGISTRY LLC, New York, NY (US)

(21) Appl. No.: 16/748,336

(22) Filed: Jan. 21, 2020

Related U.S. Application Data
(60) Provisional application No. 62/794,063, filed on Jan. 18, 2019.

(51) Int. Cl. A61B 5/15

(52) U.S. Cl. CPC A61B 5/150038; A61B 5/150366; A61B 5/1433

(57) ABSTRACT

A device and method for the collection of blood from an umbilical cord. Unlike other devices traditionally used which make use of a needle for insertion through the cord tissue and into the umbilical vein, the present device includes a long probe to be inserted into the umbilical vein after the umbilical cord is cut. The device additionally includes a fastening component to be tied around the umbilical cord under the position of the head of the device once inserted to fasten it in place. The device has a wide aperture for faster flow of the blood at a constantly tapered angle to reduce shear forces that may damage cells. Additionally, the head of the device is angled for easy insertion into the umbilical vein and is ribbed to resist falling out of the vein.

### CROSS-REFERENCE TO RELATED APPLICATION

The present application claims priority to and the benefit of U.S. Provisional Patent Application No. 62/794,063, filed on Jan. 18, 2019 and entitled “A Device For the Improved Collection of Cord Blood From an Umbilical Cord,” the entirety of which is incorporated herein by reference.

### BACKGROUND OF THE INVENTION

### 1. Field of the Invention

The present disclosure is directed generally to medical devices and cord blood banking and, more particularly, to a device for improved cord blood collection.

### 2. Description of Related Art

Collection of blood from the umbilical cord has been practiced more frequently and increased dramatically when it was found that high percentages of hematopoietic stem cells (HSCs) reside in this material previously considered as biological waste. This has sparked the emergence of a world-wide industry to collect cord blood at the time of birth, both for matching between donors and recipients and for private biobanking for family use. Standardized product lines of equipment for the collection and processing of cord blood has also emerged to support this new field.

Common devices for collection of cord blood include a needle for insertion into the vein of the umbilical cord after the cord has been clamped and cut from the newborn child directly after birth, tubing to connect this needle to a collection bag, and a collection bag pre-filled with an anticoagulant such as heparin or CPD. While these devices are well known in the art and commonly used with great success, they are not without issues.

The use of cord blood as a therapeutic has been primarily hampered by cord blood collection volumes. While cord blood can be used to treat a wide variety of conditions due to the relatively high percentage of hematopoietic stem cells, the number of stem cells needed to treat a patient is dependent on the weight of the patient, meaning larger patients require larger volumes of cord blood. Exact dosages vary by the agency recommending the dose as well as the condition being treated, but the dosage is often in the range of approximately 2.5×10\hat{ }7 TNC required per kilogram body weight of the patient receiving the cord blood. Because of this, public cord blood banks in the US have a minimum threshold of 9×10\hat{ }8 TNC for a cord blood unit to be accepted for processing and storage, and even with this threshold only the 15% of cord blood units with the highest TNC are released for therapeutic use from public banks.

While there are existing and emerging devices and processes in the art to improve TNC counts through the cord blood processing workflow so as to optimize the number of units which will realistically be used in patients, the most important factor in determining if a unit will have an acceptable TNC is the volume of cord blood actually collected when the baby is delivered. Several factors affect this volume including variables over which there is no control, such as the size of the baby at time of birth. A large factor that can be influenced is the collection itself.

Cord blood is typically collected by a physician or nurse in the delivery room at the time of birth. Many cases of a low collection of cord blood volume is not due to a lack of blood available in the cord for collection, but rather a lack of attention paid to the collection by the physician or nurse performing the collection, due to their attention being on the health of the mother and child. In these cases, the person performing the collection either may have started the collection after the placenta has been delivered (also called afterbirth), in which case the placenta stops ceases pulsing blood through the umbilical cord reducing the volume pushed to the collection needle, or the collector will take enough cord blood only to visually obtain a red color in the cord blood collection bag without attempting to collect the full volume available. As attention is, therefore, a major concern for the outcome of the collection, a system for collecting cord blood which requires minimal attention is highly desirable.

Current cord blood collection devices also present issues in several other aspects. In one aspect, the needles used in current systems are a hazard to both patients and collectors. Any time a needle is used there exists a chance for accidental needle sticks, which could potentially hurt the mother, the child, or the collector of the cord blood. More serious than the pain of an accidental needle stick is the risk for disease transmission. If, for example, the cord blood is positive for a pathogen such as HIV or HBV, an accidental needle stick could transmit such a pathogen to the collector or other person who is the victim of the accidental stick.

Another concern exists with current collection devices in which shear flow generated by high pressure of blood in the umbilical cord entering the small aperture of the collection needle can damage cells in the blood, which may include damage to the HSCs. It is therefore additionally desirable for a cord blood collection system to minimize shear flow forces in order to maximize cell viability.

Therefore, there is a need for a device to address issues of cord blood collection volumes, ease of collection, and viability of cells collected.

Description of the Related Art Section Disclaimer: To the extent that specific patents/publications/products are discussed above in this Description of the Related Art Section or elsewhere in this disclosure, these discussions should not be taken as an admission that the discussed patents/publications/products are prior art for patent law purposes. For example, some or all of the discussed patents/publications/products may not be sufficiently early in time, may not reflect subject matter developed early enough in time and/or may not be sufficiently enabling so as to amount to prior art for patent law purposes. To the extent that specific patents/publications/products are discussed above in this Description of the Related Art Section and/or throughout the application, the descriptions/disclosures of which are all hereby incorporated by reference into this document in their respective entirety(ies).

### BRIEF SUMMARY OF THE INVENTION

Embodiments of the present invention are directed to a device for improved cord blood collection and a method of cord blood collection. According to one aspect, the device includes a proximal end and a distal end with a shaft extending therebetween. The device also includes a conical shaped head at the distal end of the shaft. The conical shaped head is tapered, increasing in diameter toward the proximal end. The device additionally includes a first opening at the distal end of the conical shaped head having a first diameter, a second opening at the proximal end of the shaft having a second diameter, and an inner volume extending from the first opening and through the shaft to the second opening. The shaft is tapered toward the proximal end such that the second diameter is smaller than the first diameter.

According to another aspect, the method for collecting cord blood includes the steps of: (i) providing a collection device comprising a proximal end and a distal end with a shaft extending therebetween, a conical shaped head at the distal end of the shaft, wherein the conical shaped head is tapered, increasing in diameter toward the proximal end, a first opening at the distal end of the conical shaped head having a first diameter, a second opening at the proximal end of the shaft having a second diameter, an inner volume extending from the first opening and through the shaft to the second opening, wherein the shaft is tapered toward the proximal end such that the second diameter is smaller than the first diameter; (ii) clamping an umbilical cord with a clamp; (iii) cutting the umbilical cord at least 0.5 inches from the clamp, creating a cut end of the umbilical cord; (iv) sterilizing the cut end of the umbilical cord with an antiseptic; (v) inserting the conical shaped head into an umbilical vein up to the clamp; (vi) releasing the clamp; (vii) inserting the collection device into the umbilical vein beyond the clamp; and (viii) securing the collection device inside the umbilical vein.

These and other aspects of the invention will be apparent from and elucidated with reference to the embodiment(s) described hereinafter.

### BRIEF DESCRIPTION OF THE DRAWING

The present invention will be more fully understood and appreciated by reading the following Detailed Description in conjunction with the accompanying drawings. The accompanying drawings illustrate only typical embodiments of the disclosed subject matter and are therefore not to be considered limiting of its scope, for the disclosed subject matter may admit to other equally effective embodiments. Reference is now made briefly to the accompanying drawings, in which:

FIG. 1 is a side sectional view of a collection device, according to an embodiment; and

FIG. 2 is a side sectional view of a collection device, according to an alternative embodiment.

### DETAILED DESCRIPTION OF THE INVENTION

Aspects of the present invention and certain features, advantages, and details thereof, are explained more fully below with reference to the non-limiting examples illustrated in the accompanying drawings. Descriptions of well-known structures are omitted so as not to unnecessarily obscure the invention in detail. It should be understood, however, that the detailed description and the specific non-limiting examples, while indicating aspects of the invention, are given by way of illustration only, and are not by way of limitation. Various substitutions, modifications, additions, and/or arrangements, within the spirit and/or scope of the underlying inventive concepts will be apparent to those skilled in the art from this disclosure.

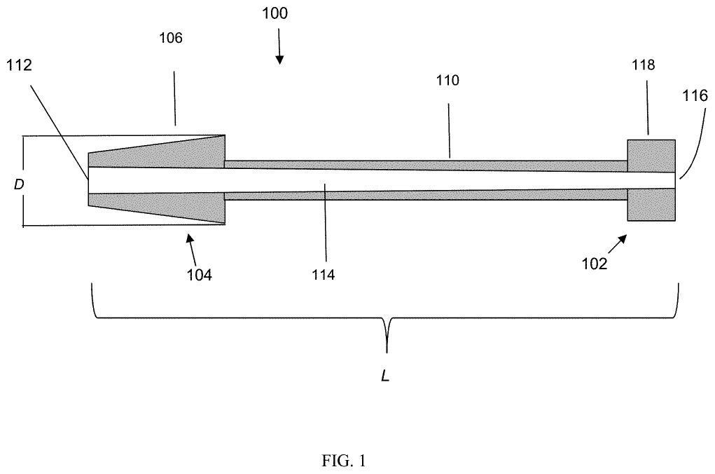

Referring now to FIG. 1 there is shown a side sectional view of a collection device 10, according to an embodiment. The present invention is a device 100 for the collection of cord blood by direct insertion into the umbilical vein after the umbilical cord is cut. The collection device 100 comprises a proximal end 102 and a distal end 104 with a cannulated shaft 110 extending therebetween. In an embodiment, the collection device 100 has a length L within the range of 1 inch to 3 inches.

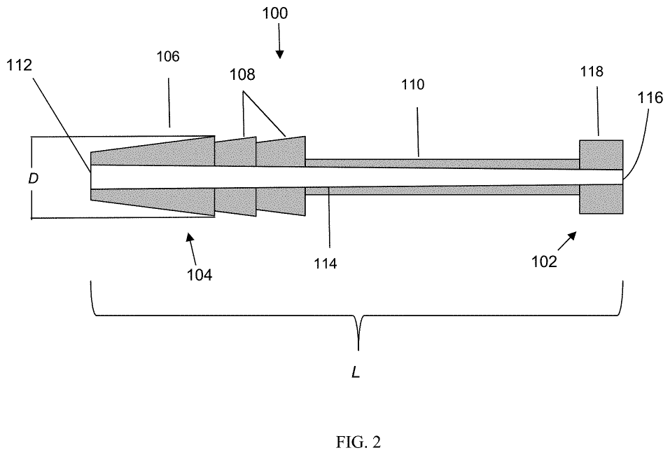

The distal end 104 of the collection device 100 comprises a conical shaped head 106 for easier insertion into the umbilical vein and to resist falling out of the vein. The conical shaped head 106 is tapered, increasing in diameter toward the proximal end 102. The conical shaped head 106 has a widest diameter D within the range of 7.5 mm to 15 mm. As shown in FIG. 1, the conical shaped head 106 is a single cone. However, in a preferred embodiment, the conical shaped head 106 is ribbed, as shown in FIG. 2. In an embodiment, the conical shaped head 106 has 2-10 ribs 108. As shown in FIG. 2, the ribs 108 extend from the conical shaped head 106 toward the proximal end 102 of the collection device 100 and extend radially (around) and from the shaft 110. The ribs 108 further inhibit the collection device 100 from falling out of the umbilical vein.

The conical shaped head 106 has a first opening 112 at a distal end 104. The first opening 112 has a diameter within the range of 3 mm to 5 mm. The shaft 110 has a diameter within the range of 4 mm to 6 mm. The shaft 110 is tapered at a constant angle toward the proximal end 102 of the collection device 100 to reduce shear forces that may damage cells. In the depicted embodiment, the shaft 110 decreases in diameter from the distal end 104 to the proximal end 102. The collection device 100 has an inner volume 114 extending from the first opening 112 and through the shaft 110 to a second opening 116 at the proximal end 102 of the collection device 100. In an embodiment, the second opening 116 has a diameter of 2 mm.

In the embodiment shown in FIGS. 1 and 2, the collection device 100 further comprises a standard luer lock 118 at its proximal end 102. The standard luer lock 118 is used for attaching the collection device 100 to cord blood collection bags. In an alternative embodiment (not shown), the collection device 100 is directly connected via medical tubing to a cord blood collection bag pre-filled with anticoagulant chosen from heparin, CPD, CDPA-1, CPDA-2, or EDTA. Accordingly, the alternative embodiment of the collection device 100 comprises a smooth proximal end 102 without the standard luer lock 118. The medical tubing has a sampling port spaced from the connection to the collection device 100 for sampling the cord blood for testing purposes. The collection device 100, according to any of the aforementioned embodiments, may additionally be used with a cable tie (not shown). The cable tie is used to secure the collection device 100 in the umbilical vein once inserted.

As stated above, in use, the collection device 100 collects blood from an umbilical cord. Upon delivery of the child whose cord blood is to be collected, the umbilical cord is clamped and cut leaving approximately at least 0.5 inch of umbilical cord after the clamp, separating the child from the umbilical cord. In one aspect, the child is delivered vaginally. In another aspect, the child is delivered by cesarean section. The cut edge of the umbilical cord is sterilized with an antiseptic. Such antiseptic can include, but is not limited to, an alcohol, such as ethanol or isopropanol, an antibiotic, an antifungal, an antiviral, a chlorine containing compound such as chlorhexidine gluconate or sodium hypochlorite, an iodine containing compound such as an iodophore, or hydrogen peroxide.

Next, the collection device 100 is inserted into the umbilical vein where the umbilical cord has been cut up to where the clamp resides. While firmly holding the collection device 100 in the vein, the clamp is removed and collection device 100 is then further inserted into the umbilical cord beyond the point of clamping. A cable tie (not shown) is then secured around the umbilical cord above where the cord had been clamped, but below where the conical shaped head 106 of the collection device 100 resides in the vein, securing the collection device 100 in the umbilical cord. The cord blood is then allowed to flow through the inner volume 114 of the collection device 100 (toward the proximal end 102 and through the second opening 116) and into the cord blood collection bag. In one aspect, this process takes place while the placenta is still in utero (i.e., during a cesarean section). In a second aspect, this process takes place after the placenta has been delivered (i.e., during a vaginal birth).

While embodiments of the present invention have been particularly shown and described with reference to certain exemplary embodiments, it will be understood by one skilled in the art that various changes in detail may be affected therein without departing from the spirit and scope of the invention as defined by claims that can be supported by the written description and drawings. Further, where exemplary embodiments are described with reference to a certain number of elements it will be understood that the exemplary embodiments can be practiced utilizing either less than or more than the certain number of elements.

What is claimed is:

1. A device for the collection of cord blood, comprising:
a proximal end and a distal end with a shaft extending therebetween;
a conical shaped head at the distal end of the shaft, wherein the conical shaped head is tapered, increasing in diameter toward the proximal end;
a first opening at the distal end of the conical shaped head having a first diameter;
a second opening at the proximal end of the shaft having a second diameter;
an inner volume extending from the first opening and through the shaft to the second opening;
wherein the shaft is tapered toward the proximal end such that the second diameter is smaller than the first diameter.

2. The device of claim 1, wherein the inner volume of the shaft is tapered at a constant angle toward the proximal end of the shaft.

3. The device of claim 1, wherein the second opening has a diameter of 2 mm.

4. The device of claim 1, wherein the conical shaped head comprises one or more ribs extending around the shaft.

5. The device of claim 4, wherein the one or more ribs are proximal relative to the conical shaped head.

6. The device of claim 4, wherein the conical shaped head has between 2 to 10 ribs.

7. The device of claim 1, further comprising a standard luer lock at the proximal end.

8. The device of claim 7, wherein the standard luer lock is directly connected to a tube with a sampling port.

9. The device of claim 7, wherein the standard luer lock is directly connected to a collection bag containing an anticoagulant.

10. The device of claim 9, wherein the anticoagulant is at least one of heparin, CPD, CPDA-1, CPDA-2, and EDTA.

11. The device of claim 1, wherein a length of the device is within the range of 1 inch to 3 inches.

12. The device of claim 1, wherein the conical shaped head has a widest diameter within the range of 7.5 mm to 15 mm.

13. The device of claim 1, wherein the shaft has a diameter within the range of 4 mm to 6 mm.

14. The device of claim 1, wherein the first opening has a diameter within the range of 3 mm to 5 mm.

15. A method for collecting cord blood, comprising the steps of:
providing a collection device comprising a proximal end and a distal end with a shaft extending therebetween, a conical shaped head at the distal end of the shaft, wherein the conical shaped head is tapered, increasing in diameter toward the proximal end, a first opening at the distal end of the conical shaped head having a first diameter, a second opening at the proximal end of the shaft having a second diameter, an inner volume extending from the first opening and through the shaft to the second opening, wherein the shaft is tapered toward the proximal end such that the second diameter is smaller than the first diameter;
clamping an umbilical cord with a clamp;
cutting the umbilical cord at least 0.5 inches from the clamp, creating a cut end of the umbilical cord;
sterilizing the cut end of the umbilical cord with an antiseptic;
inserting the conical shaped head into an umbilical vein up to the clamp;
releasing the clamp;
inserting the collection device into the umbilical vein beyond the clamp; and
securing the collection device inside the umbilical vein.

16. The method of claim 15, wherein the step of securing the collection device inside the umbilical vein includes the step of tightening a cable tie around the umbilical cord above clamp and below the conical head inside the umbilical vein.

17. The method of claim 15, wherein a placenta connected to the umbilical cord is in utero.

18. The method of claim 15, wherein a placenta connected to the umbilical cord is delivered.
